(19) United States
(12) Patent Application Publication
(10) Pub. No.: US 2011/0270140 A1
(43) Pub. Date: Nov. 3, 2011

(54) METHOD AND DEVICE FOR LESSENING THE EFFECTS OF BENIGN PROSTATIC HYPERPLASIA

(76) Inventor: Eran Israeli, Atzmon (IL)

(21) Appl. No.: 13/142,422

(22) PCT Filed: Feb. 24, 2009
(86) PCT No.: PCT/IL09/00206

§ 371 Date: Jun. 27, 2011

Related U.S. Application Data
(60) Provisional application No. 61/146,707, filed on Jan. 23, 2009.

(51) Int. Cl. A61H 1/00; A61N 1/36

(52) U.S. Cl. 601/15; 607/40; 601/46

(57) ABSTRACT

Disclosed is a method for lessening the effect of Benign Prostatic Hyperplasia by inducing increased urine flow and bladder emptying by a device including a plurality of protrusions configured to produce at least one stimulating effect on a predefined area of a foot.

### CROSS-REFERENCE TO RELATED APPLICATIONS

This application is being filed under 35 U.S.C 371 as a national patent application based on International Application Number PCT/IL2009/000206 filed on Feb. 24, 2009 which application claims priority to U.S. Provisional Application for Patent 61/146,707 filed on Jan. 23, 2009, each of which are hereby incorporated by reference, and as such this application maintains a priority date of Jan. 23, 2009.

### TECHNICAL FIELD

The method and device relate to the field of treatments for Benign Prostatic Hyperplasia, and more specifically to lessening the effect of Benign Prostatic Hyperplasia symptoms.

### BACKGROUND

Benign Prostatic Hyperplasia (BPH) affects at least 50% of men over the age of 50 years of age. The symptoms include reduced urine flow, urinary hesitancy, frequent urination, incomplete bladder emptying (urine retention), difficulty in bladder emptying and increased risk of urinary tract infections.

BPH refers to the increase in size of the prostate in middle-aged and elderly men. It is characterized by the formation of large nodules in the region of the prostate surrounding the urethral canal. The nodules compress the urethral canal to cause partial, or sometimes virtually complete, obstruction of the urethra, which interferes with the normal flow of urine. This leads to the above described symptoms.

While medication is often prescribed as the first treatment option for those suffering from clinically significant BPH, there are many patients who do not achieve success with this line of treatment. For others, though medication may be a good option, they may not achieve sustained improvement in symptoms or they may stop taking the medication because of side effects.

Patients suffering from clinically significant BPH who elect to avoid medication treatment or are non-responsive to such medication sometimes require painful surgical intervention.

Patients with minor BPH are not treated medically or surgically though still suffer from the symptoms.

Reflexology (zone therapy) is the practice of stimulating, massaging, squeezing, or pushing on predefined areas of the foot, with the goal of encouraging a beneficial effect on other parts of the body, or to improve general health.

According to Reflexology, predefined areas on the tarsal aspect of the foot correspond to specific body organs. By stimulating these predefined areas on the foot, one can induce a beneficial effect on the corresponding body organs. For example, in one exemplary embodiment of the present method, predefined areas of the foot, corresponding to the bladder and urethral canal, are stimulated. This stimulation affects the bladder and the urethral canal and induces increased urine flow through the urethral canal and bladder emptying, thus lessening the effect of BPH.

There is a need for a solution providing relief from the symptoms and lessening the effect of BPH by enabling increased urine flow and bladder emptying, which is painless, non-invasive and non-medicating. There is also a need for a solution providing relief from the symptoms for patients with minor BPH who are not treated medically or surgically though still suffer from the symptoms.

The present method and device provide a solution, alternative to medication or surgery, for such patients seeking relief from the symptoms of BPH.

### BRIEF SUMMARY

The present method and device provide an in-home self-treating solution, alternative to medication or surgery, which lessens the effect of Benign Prostatic Hyperplasia by inducing increased urine flow and bladder emptying.

### BRIEF LIST OF DRAWING

The method and the device are particularly pointed out and distinctly claimed in the concluding portion of the specification. The method and the device, however, both as to organization and method of operation, may best be understood by reference to the following detailed description when read with the accompanying drawings, in which like reference characters refer to the same parts throughout the different views. The drawings are not necessarily to scale, emphasis instead being placed upon illustrating the principles of the method.

FIG. 1 is a schematic illustration of a Reflexology map of a tarsal aspect of a foot.

FIG. 2 is a top view of an exemplary embodiment of the present device for lessening the effect of Benign Prostatic Hyperplasia by inducing increased urine flow and bladder emptying.

FIG. 3 is an oblique elevational view of an exemplary embodiment of the present device for lessening the effect of Benign Prostatic Hyperplasia by inducing increased urine flow and bladder emptying.

FIG. 4 is a schematic illustration of an exemplary embodiment of the present device for lessening the effect of Benign Prostatic Hyperplasia by inducing increased urine flow and bladder emptying.

FIG. 5 is a schematic illustration of an exemplary embodiment of the user interface for the present device for lessening the effect of Benign Prostatic Hyperplasia by inducing increased urine flow and bladder emptying.

### DETAILED DESCRIPTION OF EXEMPLARY EMBODIMENTS

In the following detailed description, reference is made to the accompanying drawings that form a part hereof and wherein like reference numerals denote like elements through the several views.

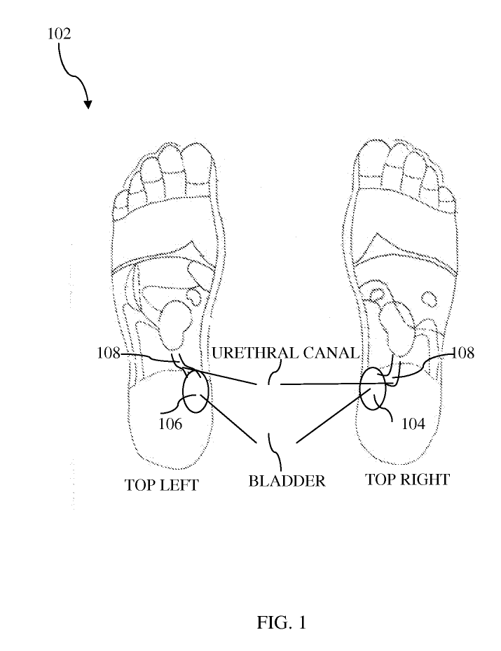

FIG. 1 illustrates a reflexology map 102 displaying the tarsal aspect (bottom) of a foot. The map outlines predefined areas corresponding to the bladder 106 and urethral canal 108 and labels 104 that specify the body organs that are affected by stimulation of these outlined predefined areas. According to Reflexology, stimulation of predefined area 106 will induce increasing bladder emptying, and stimulation of predefined area 108 will induce increased urine flow through the urethral canal thus lessening the effects of BPH.

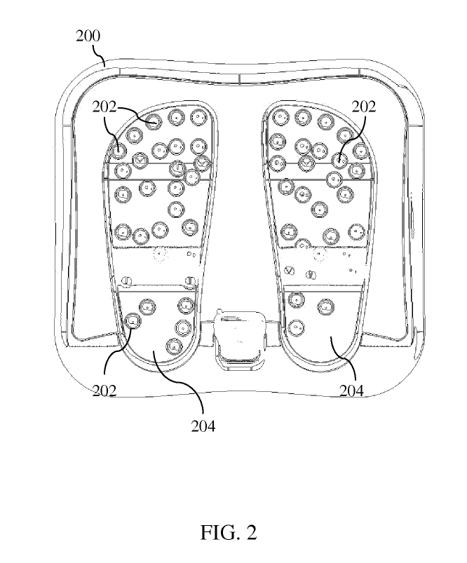

FIG. 2 illustrates a top view of an exemplary embodiment of the present device for inducing increased urine flow and bladder emptying and lessening the effect of Benign Prostatic Hyperplasia. The device 200 includes at least one plate 204 containing a plurality of protrusions 202 configured to produce at least one stimulating effect on a predefined area of a foot.

In another embodiment of the present device the plurality of protrusions 202 produce at least one stimulating effect selected from a group consisting of heating stimulation, vibrating stimulation, electric charge stimulation, electromagnetic stimulation and pressure stimulation on said foot in a pattern according to a predetermined protocol.

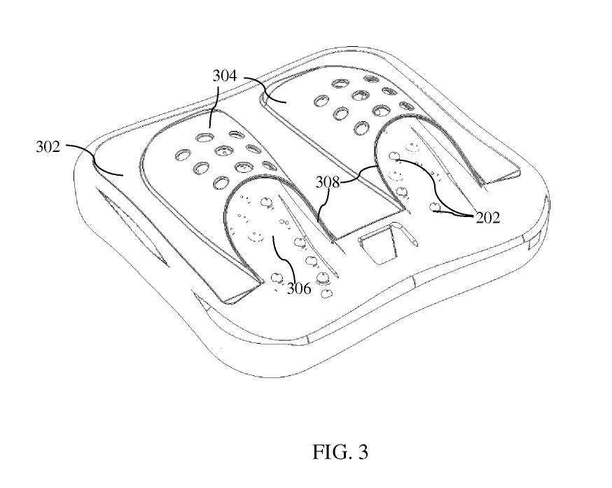

FIG. 3 illustrates an oblique elevational view of an exemplary embodiment of the present device housing 302. The housing 302 contains at least one oblong chamber 304. According to the exemplary embodiment of the device, the chamber 304 contains at least one opening 308 configured to accommodate a human foot and maintain the foot in a constant position relative to the protrusions 202. A chamber floor 306 contains a plurality of protrusions 202 configured to produce at least one stimulating effect on a predefined area of the foot in a pattern according to a predetermined protocol.

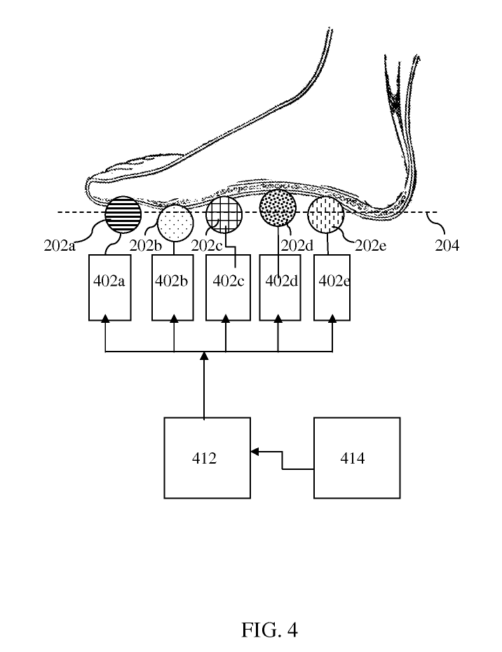

FIG. 4 is a schematic illustration of another exemplary embodiment of the device. The illustration shows a human foot placed on a plurality of protrusions 202. The axial cross section of the protrusions is of any second or higher degree curve. In the present exemplary embodiment illustrated in FIG. 4 the protrusions are spherical.

Actuators, illustrated in FIG. 4, are selected from a group consisting of a vibrational actuator, a mechanical actuator and an electromagnetic actuator, directly connected to at least one of the plurality of protrusions and configured to communicate with a control unit 412.

In still another illustrated embodiment of the present device, for example, a vibrational actuator 402a is directly connected to at least one protrusion 202a, which is a moveably attached to plate 204 and configured to communicate with the control unit 412. The vibrational actuator, when activated, causes the protrusion to vibrate along a predetermined axis according to a predetermined protocol. A mechanical actuator 402b is directly connected to at least one protrusion 202b which is moveably attached to plate 204 and configured to communicate with the control unit 412. The mechanical actuator, when activated, causes the protrusion 202b to move axially from a resting position and protrude further beyond the plane of plate 204 thus placing pressure on a predetermined area of a foot resting on protrusion 202b. The electromagnetic actuator 402c is directly connected to protrusion 202c and configured to communicate with the control unit 412.

Yet another exemplary embodiment of the present device, illustrated in FIG. 4, contains a heat source 402d which is directly connected to at least one of the plurality of protrusions 202d and configured to communicate with the control unit 412. When activated, the heat source 402d heats the connected protrusion 202d to a predetermined temperature and stimulates a predefined area of a foot resting on protrusion 202d.

Still another exemplary embodiment of the present device contains a source of electricity 402e directly connected to at least one of the plurality of protrusions 202e and configured to communicate with the control unit 412. The source of electricity 402e, when activated, is configured to cause the connected protrusion 202e to produce an electric pulse thus electrically stimulating a predefined area of a foot resting on protrusion 202e. The electric pulse current and voltage levels produced by the source of electricity are configured to be safe for application and at-home use by humans.

Still referring to FIG. 4, another exemplary embodiment of the present device contains a command unit 414 operable configured to activate the device and communicate with the plurality of protrusions 202 and the control unit 412. In yet another exemplary embodiment of the device, the command unit 414 remotely communicates with the plurality of protrusions 202 and the control unit 412.

The command unit 414 and the control unit 412 activate any one of actuators 402a-402e and corresponding protrusions 202a-202e separately, or in combination, according to a predetermined protocol, and cause a stimulating effect on predefined areas of the foot that affect the urethral canal by inducing increased urine flow and the bladder by increasing bladder emptying, thus lessening the effect of Benign Prostatic Hyperplasia.

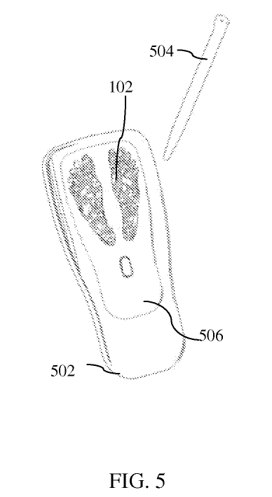

FIG. 5 is a schematic illustration of an exemplary embodiment of the command unit for the present device. In the illustrated embodiment of the device, the command unit 502 contains a touch-screen display 506 of a reflexology map 102 of a tarsal aspect (bottom) of a foot and a stylus 504 and remotely communicates with the plurality of protrusions 202 and control unit 412.

An exemplary embodiment of the present method teaches placing the foot of a subject on the plurality of protrusions 202 (See FIG. 2) and activating them to produce at least one stimulating effect selected from a group consisting of heating stimulation, vibrating stimulation, electric charge stimulation, electromagnetic stimulation and pressure stimulation on said foot in a pattern according to a predetermined protocol.

Another embodiment of the present method teaches, for example, placing the foot of a subject on the plurality of protrusions 202. Having the subject manipulate command unit 502 with stylus 504 (see FIG. 5) and touch the touch-screen display 506 to select a protocol devised to lessen the effect of Benign Prostatic Hyperplasia by inducing increased urine flow and bladder emptying. The selection is communicated to control unit 412 (See FIG. 4), which activates actuators 402a-402e and the corresponding protrusions 202a-202e, individually or in any combination, to stimulate the predefined areas on the tarsal aspect of the foot that correspond to the bladder 106 and urethral canal 108 and are in contact with the protrusions 202a-202e. Stimulating these predefined areas 106 and 108 affects the bladder and urethral canal by inducing increased urine flow and bladder emptying thus lessening the effect of Benign Prostatic Hyperplasia.

In a similar manner a number of internal diseases, constipation, and insomnia may be treated.

A number of embodiments have been described. Nevertheless, it will be understood that various modifications may be made without departing from the spirit and scope of the method. Accordingly, other embodiments are within the scope of the following claims:

What is claimed is:

1. A method for lessening the effect of Benign Prostatic Hyperplasia by inducing increased urine flow and bladder emptying, said method comprising:
providing a device including at least one plate containing a plurality of protrusions configured to produce at least one stimulating effect on a predefined area of a foot;
placing the foot of a subject on said plurality of protrusions and activating them to produce at least one stimulating effect selected from a group consisting of heating stimulation, vibrating stimulation, electric charge stimulation, electromagnetic stimulation and pressure stimulation on said foot in a pattern according to a predetermined protocol,
wherein said stimulation of said predefined area of said foot lessens the effect of Benign Prostatic Hyperplasia by inducing increased urine flow and bladder emptying.

2. The method according to claim 1, further comprising a control unit operably configured to activate said device and communicate with said protrusions.

3. The method according to claim 1, wherein at least one of said plurality of protrusions is moveably attached to said plate.

4. The method according to claim 1, further comprising a source of electricity directly connected to at least one of said plurality of protrusions.

5. The method according to claim 1, further comprising at least one actuator selected from a group consisting of a vibrational actuator, a mechanical actuator and an electromagnetic actuator, directly connected to at least one of said plurality of protrusions.

6. The method according to Claim 1, further comprising at least one heat source directly connected to at least one of said plurality of protrusions.

7. The method according to claim 1, wherein the predetermined protocol is preselected by the subject.

8. A device for lessening the effect of Benign Prostatic Hyperplasia by inducing increased urine flow and bladder emptying, said device comprising:
at least one plate containing a plurality of protrusions configured to produce at least one stimulating effect on a predefined area of a foot in a pattern according to a predetermined protocol;
a command unit operably configured to activate said device and communicate with said plurality of protrusions,
wherein said stimulating effect on a predefined area of a foot induces increased urine flow and bladder emptying and lessens the effect of Benign Prostatic Hyperplasia.

9. The device according to claim 8, wherein at least one of said plurality of protrusions is moveably attached to said plate.

10. A device for lessening the effect of Benign Prostatic Hyperplasia by inducing increased urine flow and bladder emptying, said device comprising:
at least one oblong chamber, having at least one opening and configured to accommodate at least a human foot, and a floor containing a plurality of protrusions configured to produce at least one stimulating effect on a predefined area of said foot in a pattern according to a predetermined protocol; and
a command unit operably configured to activate said device and communicate with said plurality of protrusions,
wherein said stimulating effect on a predefined area of said foot induces increased urine flow and bladder emptying and lessens the effect of Benign Prostatic Hyperplasia.

11. The device according to claim 10, wherein said device further comprises a housing configured to contain at least one oblong chamber.

12. The device according to any one of claims 8 or 10, wherein said device further comprises a control unit configured to communicate with said protrusions and said command unit.

13. The device according to any one of claims 8 or 10, wherein the axial cross section of said protrusions is of any second or higher degree curve.

14. The device according to any one of claims 8 or 10, wherein said stimulating effect is at least one effect selected from a group consisting of heating stimulation, vibrating stimulation, electric charge stimulation, electromagnetic stimulation and pressure stimulation.

15. The device according to any one of claims 8 or 10, further comprising a source of electricity directly connected to at least one of said plurality of protrusions and configured to communicate with said command unit.

16. The device according to any one of claims 8 or 10, further comprising at least one actuator selected from a group consisting of a vibrational actuator, a mechanical actuator and an electromagnetic actuator, directly connected to at least one of said plurality of protrusions.

17. The device according to any one of claims 8 or 10, further comprising at least one heat source directly connected to at least one of said plurality of protrusions and configured to communicate with said command unit.

18. The device according to any one of claims 8 or 10, wherein said command unit remotely communicates with said plurality of protrusions.

19. The device according to any one of claims 8 or 10, wherein said command unit further comprises a touch-screen display.
